(19) United States
(12) Patent Application Publication
(10) Pub. No.: US 2006/0223790 A1
(43) Pub. Date: Oct. 5, 2006

(54) Modulation of Microglial by Nicotinic Medications

(75) Inventors: Doug Shytle, Lutz, FL (US); Jun Tan, Tampa, FL (US); Francisco Fernandez, Tampa, FL (US)

Correspondence Address: SMITH HOPEN, PA, 180 PINE AVENUE NORTH, OLDSMAR, FL, 34677, US

(73) Assignee: University of South Florida, Tampa, FL (US)

(21) Appl. No.: 11/380,223

(22) Filed: Apr. 26, 2006

(30) Foreign Application Priority Data
Nov. 1, 2004 (WO) PCT/US04/36208

(51) Int. Cl. A61K 31/55; A61K 31/4439

(52) U.S. Cl. 514/214.03; 514/343

(57) ABSTRACT

A method of treating a subject suffering from a neurodegenerative disease by modulating microglial activation with a therapeutically effective amount of a cholinergic agonist and a cholinesterase inhibitor. In one embodiment of the invention, the cholinergic agonist is nicotine and the cholinesterase is galantamine (a relatively weak acetylcholinesterase inhibitor and a potent allosteric potentiating ligand of nAChRs)

### CROSS-REFERENCE TO RELATED DISCLOSURES

This application claims the benefit of international patent application number PCT/US2004/036208, filed Nov. 1, 2004, which claims the benefit of U.S. Provisional Patent Application Ser. No. 60/481,578, filed Oct. 30, 2003, which are fully incorporated herein by reference.

### BACKGROUND OF THE INVENTION

Almost all degenerative diseases of the central nervous system are associated to chronic inflammation. A central step in this process is the activation of brain mononuclear phagocyte cells, called microglia. When microglia are activated by bacterial endotoxin, lypopolysacchande (LPS), they release neurotoxic cytokines, such as Tumor Necrosis Factor (TNF-α), which causes neuronal cell death. Aβ-induced microglial activation has been proposed to contribute to neuronal dysfunction and neuronal cell death in Alzheimer's disease.

It has recently been discovered that nicotine (5 μl) and acetylcholine (10 μl) reduced microglial activation induced by bacterial endotoxin, lypopolysaccaride (LPS), an effect blocked by α7 acetylcholinergic nicotinic receptor (nAChR) antagonist, α-bungarotoxin.

Alzheimer's Disease

Because Alzheimer's disease (AD) neuropathology is characterized by β-amyloid (Aβ) plaques and neurofibrillary tangles, inhibition of Aβ accumulation is essential for effective treatment of the disease. Epidemiological studies suggest that tobacco use is associated with a reduced risk of developing AD. In addition, lower amyloid plaque densities have been observed in autopsy cortical tissue of smokers compared to non-smokers. These findings are supported by studies demonstrating neuroprotective properties of nicotine in vitro and in vivo studies demonstrating that chronic nicotine effectively reduces a peptide aggregation in the brains of Aβ overproducing mice.

Alzheimer's disease (AD) is the most common form of dementia. Deposits of the β-amyloid peptide (Aβ), produced as a cleavage product of the amyloid precursor protein (APP), are a neuropathological hallmark of AD. The ‘Swedish’ double mutation at codon 670/671 on the APP gene on chromosome 21, results in an overexpression of Aβ causing early onset familial AD. Transgenic mice, APPsw, carrying this human mutation show early phenotypic changes consistent with AD pathology, including Aβ plaque formation associated with memory deficits. This APP animal model and others are used to investigate compounds that may inhibit the Aβ accumulation and provide new treatment strategies in AD.

Aβ deposition in APP transgenic mice is reduced by a variety of interventions, including treatment with the anti-inflammatory agent, ibuprofen, clioquinol, a copper-zinc chelator, curcumin, a phyto-antioxidant, wortmannin, a phosphatidyl-inositol kinase inhibitor and vaccination with Aβ peptide. In studies in which cognitive function was assessed, reduced Aβ peptide accumulation was paralleled by attenuation of cognitive deficits.

Over the last five years there has been a rapid accumulation of evidence suggesting that neuronal nicotinic acetyicholine receptors (nAChRs) play important roles in neurodegenerative diseases. Although epidemiological studies of tobacco use and risk of developing AD are somewhat contradictory a lower amyloid plaque density has been observed in autopsy cortical tissue of smokers compared to non-smokers. In vitro studies have also indicated protective effects of nicotine against Aβ deposition and its toxicity.

HIV-associated Dementia (HAD)

HIV-associated dementia (HAD) is present in approximately 20% of AIDS patients late in the course of HIV-infection. Central to its pathological mechanism is the prolonged activation of brain mononuclear phagocytes, called microglia. Microglia provide structural and functional support to neurons as wells as serving as the primary source of productive infection by HIV-1 in the central nervous system.

Postmortem studies have suggested the brain is a viral reservoir in both demented and non-demented HIV-patients and that only those individuals with immune activation in the brain will likely develop HAD. Early in HIV infection, the infected monocyte-derived cells infiltrate the brain and HIV is observed to gain access to T-cells, microglia, and other cells primarily through interaction of gp120 binding to CD4 and the CRR5. The risk, however, of developing HAS increases late in HIV infection, as the CD4 cell count reaches 200 and below. At this time the HIV typically displays greater usage of the CXCR4 co-receptors rather than the CRR5. A CD4 cell count, which has declined to approximately 200, is believed to allow or facilitate autonomous brain infection by HIV.

Brain inflammation then becomes a self-potentiating cycle once the initial CD4 nadir is reached and immune mediators are released. For example, under the influence of IFN-Υ, expression of CD40 is dose-dependently enhanced by as much as 20-fold; greatly enhancing the ability of brain microglia to become activated. Additionally, the HIV-1 proteins, such as gp120, have been shown in vivo to be toxic to neurons via independent, direct activation of microglia.

Nicotine binding at microglial or neuronal nicotinic acetylcholine receptors (nAChR) has shown anti-inflammatory properties. Nicotine and acetylcholine inhibit LPS-induced TNF-α release in murine derived microglia. In nicotine pre-cultured neurons, binding at the α7 nAChR provides neuro-protection from the excitatory amino acid (EAA) glutamate as well as other inflammatory factors.

While nicotine's therapeutic potential is limited by pronounced nAChR desensitization following receptor activation, pharmacological approaches which improve nicotine's potency and efficacy while minimizing side effects result in significant advances in the treatment of neuroinflammatory disorders, such as AD.

Galantamine, a weak acetylcholinesterase inhibitor and a potent allosteric potentiating ligand of nAChRs, is an approved and effective treatment for Alzheimer's disease. Galantamine allosterically potentiates agonist responses at human α7 nAChRs (studied in oocytes) in the same window of concentrations (i.e., 0.1-1.0 gM), which correlates with cerebrospinal fluid concentration of the drug at the recommended daily dosage of 16 to 24 mg. Galantamine up-regulates agonsist responses of nAChR receptors at concentrations between 0.1 and 1 μM while concentrations greater then 10 μM result in nAChR inhibition. In vivo studies have shown that galantamine, acting as an APL on preseynaptic and tonically active nAChRs, potentiates glutameric or GABAergic transmission whereas the non-APL cholinesterase inhibitors lack this therapeutic effect on synaptic transmission. Because minimal galantamine-induced acetyicholinesterase inhibition occurs in this concentration range, the therapeutic action of galantamine is produced by its sensitizing action on nAChRs rather than by general cholinergic enhancement due to cholinesterase inhibition.

### SUMMARY OF INVENTION

The inventive method of treating a subject afflicted with a neurodegenerative disorder, comprises the step of concomitantly administering a therapeutically effective amount of a cholinergic agonist and a cholinesterase inhibitor to the subject. In alternate embodiments, the neurodegenerative disorder is selected from the group consisting of HIV-associated dementia (HAD) and Alzheimer's disease (AD). Furthermore, the administration of the cholinergic agonist, such as nicotine, and acetylcholinesterase inhibitor, such as galantamine, is directed to a microglia target cell.

In different embodiments of the inventive method, nicotine is administered in concentrations of about 0.3 μM to 5 μm and galantamine is administered in concentrations of about 0.05 μM to 10 μM.

### BRIEF DESCRIPTION OF THE DRAWINGS

For a fuller understanding of the nature and objects of the invention, reference should be made to the following detailed description, taken in connection with the accompanying drawings.

FIG. 1 Total RNA are isolated from N9 microglial cell line and primary cultured microglial cells for reverse transcriptase-polymerase chain reaction (RT-PCR) analysis. Murine primary culture microglial cells are isolated from mouse cerebral cortices (C57BL/6 mice) and are grown in RPMI medium. As a result, nAChR α-7 subunit mRNA is detected in both of these cells.

FIG. 2 Nicotine and galantamine combination synergistically reduced LPS-induced TNF-α release.

FIG. 3A shows the amount of TNF-α release from microglial cell induced by HIV glycoprotein (gp120) or Interferon-gamma (IFN-Υ)or the combination of gp120/lnterferon-Υ.

FIG. 3B shows the modulation of TNF-α release from microglial cells with galantamine or galanatmine and nicotine combination.

FIG. 4 is a graph showing the synergistic effect of HIV-1 gp120 and IFN-Υ on microglial activation.

FIG. 5 shows graphs which establish that galantamine and nicotine synergistically inhibit microglial activation induced by HIV gp 120 and IFN-Υ, which is attenuated by the presence of α-bungarotoxin (alpha-Bgt), a selective α7 nicotinic agonist.

FIG. 6 shows immunoblots and graphs showing that pretreatment of cultured microglial cells with galantamine and nicotine results in inhibition of phosphorylation of p44/42 MAPK induced by HIV-1 gp120/IFN-Υ (IFN-g).

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

The studies described below in Example I demonstrate dose-response functions of galantamine, nicotine, and their combination on microglial cytokine release (TNF-α, IL-6, and IL-1β) induced by exposure to LPS can be characterized by the current invention. Secondary advantages of the present invention also disclosed in Example I include a) the discovery of the expression of other nAChR subunits and their roles in microglia modulation and b) the discovery of previously unknown downstream processes such as protein kinase phosphorylation.

The studies in Example II establish that microglial release of TNF-α and NO are positively regulated by the addition of HIV-1 gpI20 and IFN-Υ. The CXCR4 receptor is instrumental in modulating the intensity of the synergistic relationship such that there it is negatively regulated in the presence of a selective CXCR4 blockade. This provides a novel in vitro model for the study of HAD. The inventors also disclose that galantamine and nicotine, acting through α7 nAChR's p44/42 MAPK system, is a novel combination for synergistically reducing HIV mediated microglial activation.

Pharmaceutical Compositions

Modulators of microglial activity (also referred to herein as “active compounds”) can be incorporated into pharmaceutical compositions suitable for administration. Such compositions typically comprise the compound a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compounds in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from pressured container or dispenser which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Pharmacogenomics

Agents, or modulators which have a stimulatory or inhibitory effect on cholinergic activity can be administered to individuals to treat (prophylactically or therapeutically) disorders (e.g., neurological inflammation) associated with aberrant microglial activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., nicotine) for prophylactic or therapeutic treatments based on a consideration of the individual's genotype. Such pharmacogenomics can further be used to determine appropriate dosages and therapeutic regimens. Accordingly, the activity of the active compounds in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder (1997) Clin. Chem. 43(2): 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans).

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification. Thus, the activity of the active compounds in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a the target gene modulator, such as a modulator identified by one of the exemplary screening assays described herein.

The dose of the compound is that amount effective to treat the neurodegenerative disorder from which the subject suffers. By “therapeutically effective amount” is meant that amount sufficient to pass across the blood-brain barrier of the patient, to bind to relevant receptor sites in the brain of the patient, such as the α7 nAChR, and to elicit neuropharmacological effects (e.g., elicit neurotransmitter secretion, thus resulting in effective treatment of the disease). Treatment of a neurodegenerative disorder involves a decrease of symptoms of the particular disease. The compounds useful according to the method of the present invention have the ability to pass across the blood-brain barrier of the patient. As such, such compounds have the ability to enter the central nervous system of the patient. The log P values of typical compounds useful in carrying out the present invention generally are greater than 0, often are greater than about 0.1, and frequently are greater than about 0.5. The log P values of such typical compounds generally are less than about 3.0, often are less than about 2.5, and frequently are less than about 2.0. Log P values provide a measure of the ability of a compound to pass across a diffusion barrier, such as a biological membrane. See, Hansch, et al., J. Med. Chem., Vol. 11, p. 1 (1968) (incorporated herein by reference).

### EXAMPLE I

The dose-response functions of galantamine, nicotine, and their combination on microglial cytokine release (TNF-α, IL-6, and IL-1β) induced by exposure to LPS can be characterized by the current invention. Secondary advantages of the present invention include a) the discovery of the expression of other nAChR subunits and their roles in microglia modulation and b) the discovery of previously unknown downstream processes such as protein kinase phosphorylation.

Nicotinic Acetyicholine Receptor A 7 Subunit is Expressed by Microglial Cells

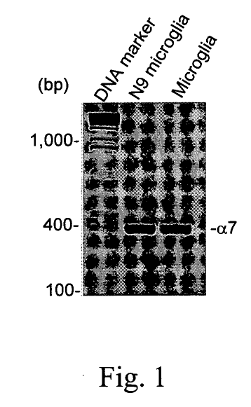

In order to investigate whether nAChR \acute{α}7 subunit is expressed in cultured microglial cells, total RNA were isolated from N9 microglial cell line and primary cultured microglial cells for reverse transcriptase-polymerase chain reaction (RT-PCR) analysis. Murine primary culture microglial cells are isolated from mouse cerebral cortices (C57BL/6 mice) and are grown in RPMI medium. As a result, nAChR α7 subunit mRNA is detected in both of these cells (FIG. 1)

Synergistic Attenuation of Microglial Activation by Galantamine and Nicotine Combination

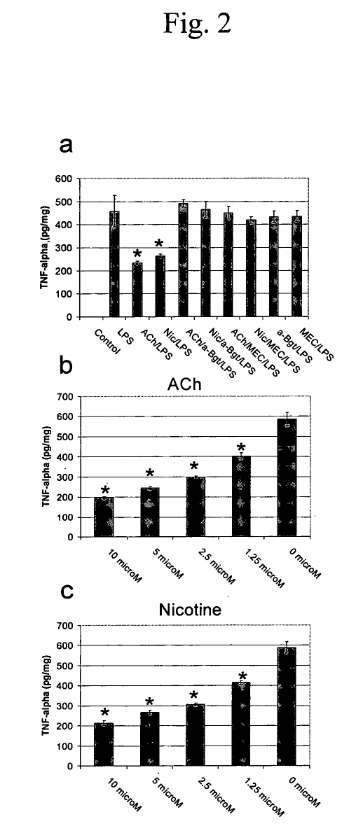

Negative regulation of microglia activation represents additional mechanism underlying nicotine's reported neuroprotective properties. Electrophysiological studies, however, suggest the nicotine's therapeutic potential is limited by pronounced nAChR desensitization following receptor activation. Therefore, pharmacological approaches which improve nicotine's potency and efficacy while minimizing side effects will result in significant advances in the treatment of neuroinflammatory disorders. Galantamine, a weak acetylcholinesterase inhibitor and a potent allosteric potentiating ligand of nAChRs, is an approved treatment for Alzheimer's disease (AD). Cultured Pre-microglial cells were pre-incubated with 0.1 μM nicotine and 0.05 μM galantamine for 30 minutes and challenged these cells with LPS for 4 hours to obtain preliminary data on nicotine and galantamine cotreatment on microglial activation. As shown in FIG. 2, nicotine and galantamine combination synergistically reduced LPS-induced TNF-a release. The co-administration of galantamine and nicotine yielded surprising results.

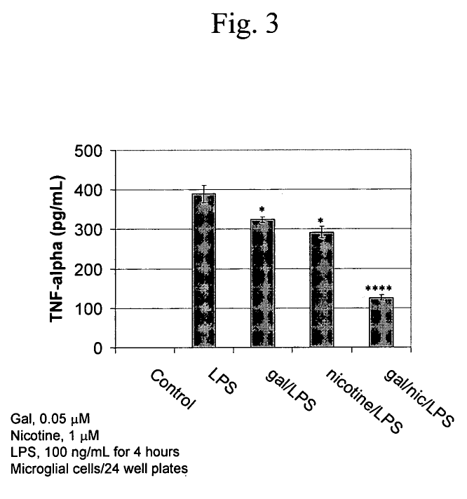

FIG. 3A shows the amount of TNF-α release from microglial cell induced by HIV glycoprotein (gp120) or Interferon-gamma (IFN-Υ)or the combination of gp120/lnterferon-Υ. FIG. 3B shows the unexpected modulation of TNF-α release from microglial cells with galantamine or galantamine and nicotine combination.

Murine Primary Microglial Cell Culture

Breeding pairs of C57BL/6 mice were purchased from Jackson Laboratory (Bar Harbor, Me.) and housed in the animal facility at the University of South Florida Health Science Center. Murine primary culture microglial cells are isolated from mouse cerebral cortices and were grown in RPMI medium according to previously described methods (See Tan et al. J. Biol Chem 275, 37224-31 (2000). Briefly, cerebral cortices from newborn mice (1-2 days old) were isolated under sterile conditions and kept at 4° C. prior to mechanical dissociation. Cells are plated in 75-cm flasks, and completed medium are added. Primary cultures are kept for 14 days so that only glial cells remain, and microglial cells are isolated by shaking flasks at 200 rpm.

ELISA

Primary cultured microglial cells were plated in 24-well tissue culture plates at 5×10⁴ cells/well and were pretreated for 60 minutes with either nicotine (0.3-3.0 μM), galantamine (0.05-0.5 μM), and their combination and were then challenged with LPS (100 ng/mL) for 4 hours. Experiments were conducted in triplicate and data combined for analyses. Cell-free supernatants were collected and assayed for TNF-α, IL-6 and IL-1μ release by ELISA.

Murine Primary Neuronal Cell Culture

Murine primary culture neuronal cells were isolated from mouse cerebral cortices and were grown in DMEM medium according to previously described methods. Briefly, cerebral cortices from newborn mice (15-17 days old in utero) were isolated under sterile conditions and kept at 4° C. prior to mechanical dissociation. Cells were plated in 24-well tissue culture plates at 2.5×10⁵ cells per well after collagen coating the plates, and completed medium were added.

Lactate Dehydrohenase Release Assay in Neuronal and Microglial Co-Cultures

After 5 days in vitro, neuronal cells were passed in prep for subsequent experiments. Following confirmation of differentiation (with neurofilament L staining), neuronal cells were seeded in 24-well tissue culture plates at 1×10⁵ cells per well for 48 hours and used as target cells for lactate dehydrogenase (LDH) release assay. Neuronal-microglial co cultures (microglia, 5×10 cells per well, a 2:1 ratio of neurons to microglia) were treated with nicotine, galantamine, or their combination followed by LPS for 4 hours. An LDH release assay was performed after 36 or 48 hours of treatment in neuronal cultures.

As stated previously the degree of β-amyloidosis was recently assessed in both immunohistochemically and biochemically in APPsw mice chronically treated with nicotine. The reduction in β-amyloid plaque observed in 14.5 month APP mice after 5.5 months of nicotine treatment was comparable to that observed in 16 month APP mice receiving 8.5 months Aβ immunization. Findings show that the combination of galantamine and nicotine chronically have a greater efficacy in reducing β-amyloidosis with less side effects than either drug given alone.

Animals

Previous studies assessed the effects of chronic nicotine in transgenic mice expressing a mutant human a chimeric mouse/human amyloid precursor protein (APPsw). However, this mouse strain does not show plaque deposits until 12-18 months of age. In the present method a newer double transgenic mouse strain expressing both a mutant human presenilin 1 (DeltaE9) and a chimeric mouse/human amyloid precursor protein (APPswe) was used. Due to the double mutation, this transgenic strain develops brain β-amyloid deposits by 6 to 7 months of age allowing for more expedient pharmacological testing.

Long-Term Nicotine and Galantamine Cotreatment

Five month old female mice overexpressing Aβ (DeltaE9/APPswe) were treated with nicotine plus sucrose (2%), galantamine plus sucrose (2%), galantamine/nicotine plus sucrose (2%) or sucrose alone for 5 months in drinking water. Nicotine was gradually increased from 25 μg/mL (free base) on day 1, to 50 μg/mL on days 2-3, 100 μg/mL on days 4-6, and 200 μg/mL thereafter. A low concentration of nicotine (50 μg/mL) along with 3 different concentrations of galantamine plus sucrose was then tested. However, the exact dose range to be used is determined. Fresh solutions were made every second day. Animals were chronically treated with nicotine, galantamine, galantamine/nicotine, or vehicle from 5 to 11 months of age before brain analyses. Forty-eight hours before killing, all treatment solutions were replaced with sucrose alone. Mice were killed by cervical dislocation. One cerebral hemisphere were fixed in 1% paraformaldehyde in phosphate buffered saline for 24 h and stored in saline with sodium azide, and the others were immediately frozen and stored at −80° C.

Biochemical Analysis of Soluble and Insoluble A/I1-40 and A/I1-42 Concentrations

Cortical tissue was homogenized in 6.5 volume of buffer containing 20 mm Tris-HCL, pH 8.5 and protease inhibitors (Complete, Scandinavia AB). After centrifugation (100,000 g for 1 h at 4° C.), the supernatant was diluted 1:1 with phosphate buffered saline including 0.5% bovine serum albumin, 0.05% Tween 20 and protease inhibitors (standard buffer) and used for analysis of soluble Aβ. The pellet was extracted in 10 volume of 5 m guanidine-HC1 in 20 mm Tris HC1, pH 8.0, diluted 1:10 with standard buffer and centrifuged at 13,100×g for 25 mm at 4° C. The supernatant was further diluted 1:25 with standard buffer plus 0.5 m guanidine-HC1. The levels of Aβ1-40 and 42 were analyzed by colorimetric sandwich ELISA kits. The absorbance at 450 nm is determined using a spectra max 250 microplate spectrophotometer.

A/l 1-42 Immunohistochemistry

Ten microns of fixed, paraffin embedded sections were rehydrated, treated for 3 mm with concentrated formic acid and then 10% hydrogen peroxide. Sections were incubated for 60 minutes with an affinity purified rabbit polyclonal antibody directed against human Aβ 1-42 (Kalaria et al. 1996), diluted 1:5000. Antigen-antibody reactions were visualized using the Vectastain Elite kit method (Vector Laboratories) and 3, 3-diaminobenzidine as chromogen. Images of whole brain wereas within parasagittal sections (3 replicates per werea) were assessed using a computerized image analyser, MCID M 5 plus system (Imaging Research mc, Brock University, Canada) attached to a light microscope (×4 objective). Plaques density is estimated as the werea of Aβ 1-42 immunoreactivity expressed as a percentage of total field. Assessments were carried out blind to the animal grouping.

GFAF Immunohistochemistry

Ten microns of paraffin embedded sections were rehydrated, treated for 3 mm with concentrated formic acid and then 10% hydrogen peroxide. Sections were incubated for 60 minutes with an affinity purified rabbit polyclonal antibody directed against GFAP using antiserum (DAKO Z 0334) diluted 1:2000. Antigen-antibody reactions were visualized using the Vectastain Elite kit method (Vector Laboratories) and 3, 3′-diaminobenzidine as chromogen. Images of whole brain wereas within parasagittal sections (3 replicates per werea) were assessed using a computerized image analyser, MCID M 5 plus system (Imaging Research mc, Brock University, Canada) attached to a light microscope (×4 objective). Plaques density were estimated as the werea of GFAP immunoreactivity expressed as a percentage of total field. Assessments were carried out blind to the animal grouping.

### EXAMPLE II

Microglial modulation by nAChRs represents a novel physiological mechanism for the reported neuroprotective properties of nicotinic drugs in animal models of neurodegenerative disease. In the peripheral nervous system, a non-neuronal cholinergic system is strongly expressed within different components of the immune system and is likely involved in the regulation of host inflammation. An example has been provided by Wang et al. Nature 421, 384-388 (2003), who have shown that efferent vagus nerve stimulation attenuates the systemic inflammatory response to LPS in blood-borne macrophages and this is mediated by acetylcholine acting at α7 nAChRs (see Wang, supra). The present invention discloses a similar role for nicotine and galantamine at the same receptor; this time involved in regulation of inflammation in the brain. Microglia can serve both neurotrophic and neurotoxic functions in the brain and factors determining which function microglia carry out depend on a combination of signals received from nearby astrocytes and neurons, Polazzi, Rev. Neurosci. 13, 221-242 (2002). The results are in agreement with this hypothesis and show that signals related to suppression of the immunological cytokine release involve neuronal cholinergic communication via α7 nAChR's in the healthy in vivo state.

In summary, microglial release of TNF-α and NO are positively regulated by the addition of HIV-1 gpI20 and IFN-Υ. The CXCR4 receptor is instrumental in modulating the intensity of the synergistic relationship such that there it is negatively regulated in the presence of a selective CXCR4 blockade. This provides a novel in vitro model for the study of HAD. The inventors also disclose that galantamine and nicotine, acting through α7 nAChR's p44/42 MAPK system, is a novel combination for synergistically reducing HIV mediated microglial activation.

Murine Primary Cell Culture

Murine primary culture microglial cells were isolated from mouse cerebral cortices and were grown in RPMI 1640 medium supplemented with 5% fetal calf serum, 2 mM glutamine, 100 U/mL penicillin, 0.1 μg/mM streptomycin, and 0.05 μM 2-mercaptoethanol according to previously described methods, Tan et al., J. Biol. Chem. 275, 37224-37231, (2000). Briefly, cerebral cortices from newborn mice (1-day-old) were insolated under sterile conditions and were kept at 4° C. before mechanical dissociation. Cells were plated in 75 cm² flasks (NunclonTM, Roskilde, Denmark), and complete medium was added. Primary cultures were kept for 2 weeks so that only glial cells remained, and microglial cells were isolated. More than 98% of these cells were stained positive for CD11b (Boehringer Mannheim, Indianapolis, Ind.).

TNF-α ELISA and Nitric Oxide Release Assay

Primary cultured microglial cells were plated in 24-well tissue culture plates (NUNCLON, Roskilde, Denmark) at 1×10⁵ cells/well. Some of these cells were treated with either HIV-1_{CN54} gpI20 (2 μg/mL; provided by NIH ADS Research & Reference Reagent Program) or murine recombinant IFN-Υ (100 ng/mL; R&D systems, Minneapolis, Minn.) or a combination of HIV- 1_{CN54} gp120 (2 μg/mL) and IFN-Υ (100 ng/mL) in the presence or absence of anti-CXCR4 antibody (2 μg/mL; Clone, 44717; provided by NIH AIDS Research & Reference Reagent Program) or control antibody (PharMingen, San Diego, Calif.) for 24 hours. Some of these cultured cells were pretreated with 0.05 μM galantamine (Sigma) and/or 5 μM nicotine (Sigma) for 30 minutes and then co-challenged with HIV- 1 gp120 (2 μg/mL) and IFN-Υ (100 ng/mL) for 8 hours in the presence or absence of a-bungarotoxin (10 nM, Sigma). Cell-free supernatants were then collected and assayed by a TNF-α ELISA kit (R&D systems) or by a nitric oxide (NO) assay kit (Calbiochem, La, Jolla, Calif.) in strict accordance with the manufacturer's instructions. The Bio-Rad protein assay (Hercules, Calif.) was performed to measure total cellular protein from each of the cell groups under consideration just prior to quantification of TNF-α release by ELISA or NO secretion by NO assay.

Western Limmunoblotting

Murine primary culture microglial cells were plated in six-well tissue culture plates (NUCLON) at a density of 1×10⁶ cells/well. As previously described in Shytle et al. J. Neurochem. 89, 337-343 (2004), for examining phosphorylation of p44/42 MAPK, these cells were co-pretreated with galantamine (0.05 μM) and nicotine (5 μM) for 30 minutes in the presence or absence of α-bugarotoxin (10 nM) and then challenged with HIV-1_{CN54} gp120 (2 jig/mL) and IFN-Υ (100 ng/mL) for various time points. Immediately following culturing, microglial cells were washed in ice-cold PBS, and lysed in an ice-cold lysis buffer. After incubating for 30 minutes on ice, samples were centrifuged at high speed for 15 minutes, and supernatants were collected. Total protein content was estimated using the Bio-Rad protein assay. An aliquot corresponding to 50 μg of total protein of each sample was separated by SDS-polyacrylamide gel electrophoresis and transferred electrophoretically to Immuno-Blot polyvinylidene difluoride membranes. Nonspecific antibody binding was blocked with 5% nonfat dry milk in Tris-buffered saline (20 mM Tris, 500 mM NaCI, pH 7,5) for 1 h at room temperature. Membranes were hybridized with phosphospecific p44/42 MAPK monoclonal antibody, stripped with 3-mercaptoethanol stripping solution (62.5 mM Tris-HCI, pH 6.8; 2% SDS, and 100 mM -mercaptoethanol), and then re-probed with an antibody that recognizes total p44/42. Alternatively, membranes with identical samples were probed with either phosphor-specific p44/42 with an antibody that recognizes total p44/42 MAPK. Immunoblotting was carried out with a primary antibody followed by an anti-rabbit or anti-mouse horseradish peroxidase (HPR)-conjugated IgG secondary antibody as a tracer. The Immuno-Star' chemiluminescence substrate was used to develop the blots.

Statistical Analysis

Data were analyzed using ANOVA followed by post hoc comparisons of means by Bonferroni's or Dunnett's T3 method, for which Levene's test for homogeneity of variances was used to determine the appropriate method of post hoc comparison. In instances of single-mean comparison, t-test for independent samples was used to assess significance. The α levels were set at 0.05 for each analysis. All analyses were performed using SPSS for Windows release 9.0.

IFN-Υ Synergistically Enhances HIV-1 gpl20-induced Microglial Activation

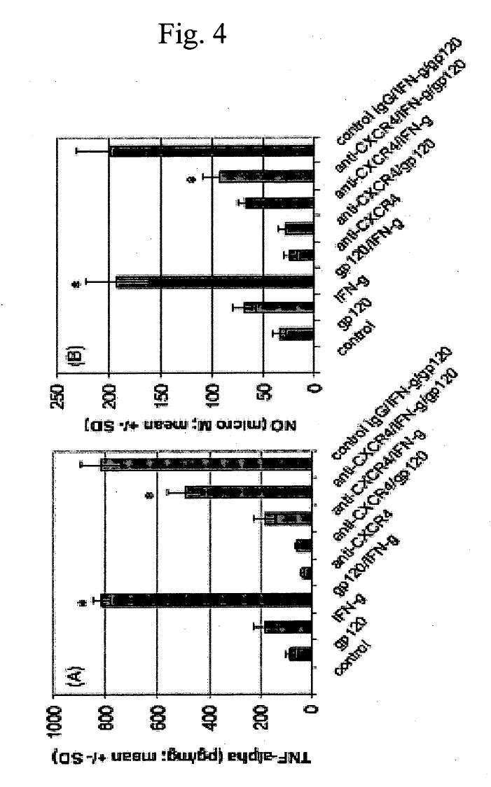

It has been previously reported that IFN-Υ has a synergistic effect on cytokine production secreted by cultured microglial cells treated with stimuli (such as amyloid β peptides) (see Tan et al., Science 286, 2352-2355 (1999)). In order to test whether IFN-Υ could synergistically enhance HIV-1 gpl20-induced microglial activation, the inventors co-treated primary cultured microglial cells with IFN-Υ and HIV1 gp120 in the presence or absence of anti-CXCR4 antibody or control antibody for 24 hours. Cell-free supernatants were collected from each of the cell groups and assayed by a TNF-α ELISA kit and by a NO release assay in strict accordance with the manufacturer's instructions. The Bio-Rad protein assay was performed to measure total cellular protein from each of the cell groups under consideration just before quantification of TNF-α production and NO release. Data show that co-treatment of cultured microglial cells with IFN-Υ and HIV-1 gp120 results in a synergistic effect on microgial activation as evidenced by increased TNF-α production (FIG. 4A) and elevated level of NO secretion (FIG. 4B). Furthermore, these effects were significantly attenuated by the presence of anti-CXCR4 antibody, but not control antibody, showing that HIV-I gpl20 specifically has an effect on microglial activation. Primary cultured microglial cells (1×10⁵ per well in 24-well tissue culture plate) were treated with HIV-1 gpl20 (2 μg/mL) or IFN-Υ (IFN-Υ; 100 ng/mL), or HIV-1/IFN-Υ in the presence or absence of anti-CXCR4 antibody (2 μg/mL) or these cells went with no treatment (control) for 24 hours. Microglial activation was measured by TNF-α production (mean±S.D., picograms per milligram of total protein) in cultured media by TNF-α ELISA (A), NO release (mean±S.D., micromolar concentration per milligram of total protein) in cultured media by NO assay (B). For (A and B), ANOVA revealed that a significant interaction between gpl20 and IFN-Υ (P<0.001) compared with either gpl20 or IFN-Υ treatment alone. Further, there is a significant difference between gpl20/lFN-Υ and anti-CXCR4/IFN-Υ/gp120 (P<0.005).

Co-treatment with Galantamine and Nicotine Synergistically Oppose HIV-1 gpl2O/IFN-Υ- Induced Microglial Activation

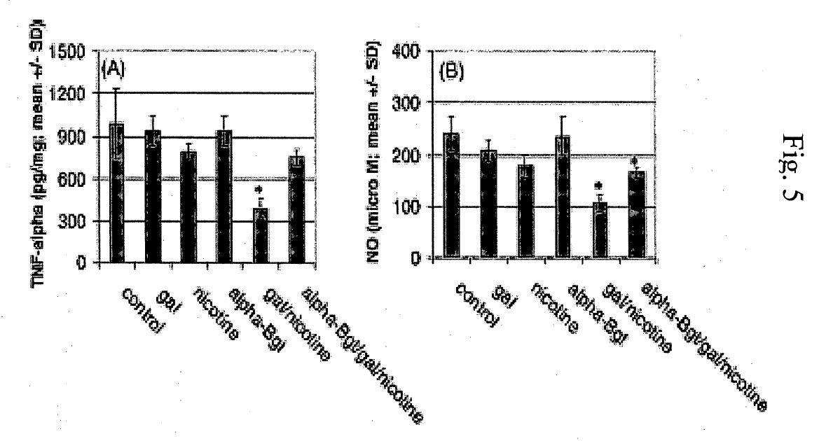

The inventor's own studies have shown that the cholinergic pathway is involved in negative regulation of microglial activation through α7 nicotinic acetylcholine receptor (nAChR) (Shytle et al.). In addition, it is well known that galantamine is a potent allosteric potentiating ligand (APL) of nAChRs (See, Samochocki et al., J. Pharmacol. Exp. Ther., 1024-1036 (2003)). In order to determine whether galantamine and/or nicotine could inhibit HIV-1 gp120/IFN-Υ-induced TNF-α production and NO release in cultured microglial cells, the inventors pre-incubated microglial cells with galantamine, nicotine, or a combination of galantamine and nicotine for 30 minutes and then challenged these cells with HIV-I gpl20/IFN-Υ for 8 hours. As shown in FIG. 5A and C, data indicate that the pretreatment with either galantamine or nicotine results in a slight reduction of HIV-1 gpl20/IFN-Υ-induced TNF-α production and NO release. Most importantly, the pretreatment with a combination of galantamine and nicotine produces an even greater marked reduction of HIV-1 gpl20/IFN-Υ-induced TNF-α production and NO release (FIG. 5A and C). Furthermore, in order to test if this effect is specifically to the action of α7 nAChR, the inventors co-pretreated these cells with galantamine and nicotine in the presence or absence of a-bungarotoxin, a selective 7 nAChR antagonist, for 30 minutes and then challenged them with HIV-1 gpl20/IFN-Υ for 8 hours. Data show that this co-pretreatment of these cells with α-bungarotoxin significantly attenuates the effect of galantamine/nicotine on inhibition of microglial TNF-α production and NO release induced by HIV-1 gpl20 and IFN-α challenge (FIG. 5B and D). Galantamine and nicotine synergistically inhibit microglial activation induced by HIV-1 gpl20 and IFN—co-treatment, which is attenuated by the presence of α-bungarotoxin (alpha-Bgt), a selective α7 nicotinic antagonist. As described in FIG. 4, primary cultured microglial cells were pretreated with galantamine (gal; 0.05 M) and/or nicotine (5 M) in the presence or absence of α-bungarotoxin (10 nM) for 30 minutes and then challenged with HIV-1 gpl20 (2 g/mL) and IFN-Υ (100 ng/mL) for 8 hours. Co-treatment of these cells with galantamine and nicotine markedly inhibits microglial activation as evidenced by decreased TNF-α production (A, mean±S.D., picograms per milligram of total protein) and reduced NO release (B, mean±S.D., micromolar concentration per milligram of total protein) in cultured media. These effects are significantly blocked by co-pretreatment of microglial cells with a-bungarotoxin (A and B). For (A and B), ANOVA revealed that significant main effects of co-treatment of these cells with galantamine and nicotine compared with control (gp120/IFN-Υ challenge alone) and gpl20/IFN-Υ challenge in the presence of either galantamine or nicotine (P<0.005). Furthermore, ANOVA revealed that a significant main effect of co-pretreatment of these cells with s-bungarotoxin compared with pretreatment of these cells with galantamine/nicotine in the absence of a-bungarotoxin (P<0.005).

Co-pretreatment with Galantamine/nicotine Suppresses HIV-1 gpl20/IFN-Υ-induced Microglial Activation Through Inhibiting Phosphorylation of p44/42 MAPK

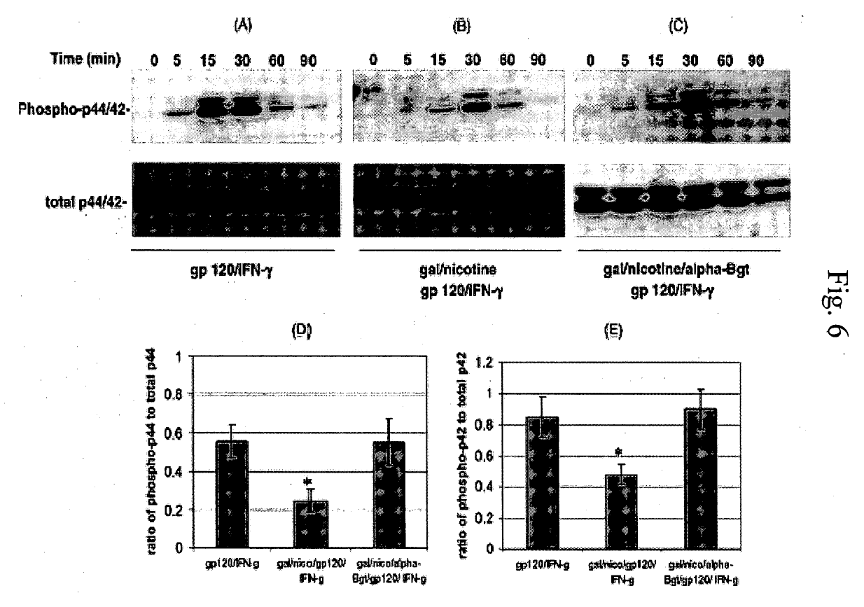

Previous studies have shown that activation of mitogen-activated protein kinase (MAPK) p44/42 is involved in TNF-α production in macrophages, monocytes and microglia after activation of these cells with a variety of stimuli, including LPS and CD4O ligand (See Tan et al., supra). Given that the combination of galantamine and nicotine at low doses greatly opposes microglial activation as evidenced by a reduction of TNF-α production and NO release, the inventors wished to determine whether reduced phosphorylation of p44/42 could be responsible for these effects. Thus, the inventors analyzed p44/42 phosphorylation status in microglial cell lysates after pretreatment with the combination of galantamine (0.05 μM) and nicotine (5 μM) for 30 minutes and then challenge with HIV- 1 gp120 IFN-Υ at a variety of time points. As shown in FIG. 6A and B, results show that pretreatment with the combination of galantamine and nicotine significantly inhibits phosphorylation of p44/42 MAPK induced by HIV-1 gpl20/IFN-Υ compared with controls (HIV-I gp120/IFN-α challenge alone), demonstrating the functionality of the combination of galantamine and nicotine co-stimulated cholinergic signaling on reduction of p44/42 MAPK activation. To further evaluate this functionality, the inventors pretreated microglial cells with the combination galantamine/nicotine in the presence of α-bungarotoxin. Thirty minutes later, these cells were challenged with HIV-1 gp120/TFN-Υ. Phosphorylation status of p44/42 MAPK was examined by western blot. As shown in FIG. 6C, this pretreatment leads to attenuating the effects of the combination galantamine and nicotine on inhibition of phosphorylation of p44/42 MAPK induced by HIV-1 gpl20/IFN-Υ. Finally, to semi-quantify these data, the inventors carried out the densitometric analysis as previously described (See Shytle et al., supra). As shown in FIG. 6D and E, the co-pretreatment of these cells with galantamine and nicotine markedly inhibits phosphorylation of p44/42 MAPK compared with gpl20/IFN-Υ challenge alone. However, this effect was significantly attenuated by the presence of α-bungarotoxin.

Pretreatment of cultured microglial cells with galantamine and nicotine results in inhibition of phosphorylation of p44/42 MAPK induced by HIV1 gpl20/IFN-Υ (IFN-Υ) (FIG. 6). Primary cultured microglial cells (1×10⁶ per well in six-well tissue culture plate) were co-pretreated with galantamine (gal) and nicotine (nico) in the presence (C) or absence (B) of α-bungarotoxin (ct-Bgt) for 30 minutes and then challenged with HIV-1 gpl20 (2 μg/mL) and IFN-Υ (100 ng/mL) or went control (A; HIV-I gpl20/IFN-Υ challenge alone) for various time points as indicated. The phosphorylation of p44/42 MAPK was measured by western blot using the antibodies specifically against phopho-p44/42 and total p44/42. Data presented here are representative of three independent experiments. Histogram represents the mean band density±S.D. (D, ratio of phospho-p44 MAPK to total p44 MAPK at 30 minutes; E, ratio of phospho-p42 MAPK to total p42 MAPK at 30 minutes). ANOVA revealed the significant main effects of co-pretreatment of these cells with galantamine and nicotine compared with control (gp120/IFN-Υ challenge alone) (P<0.001). However, there is not a significant main effect of co-pretreatment of these cells with galantamine and nicotine compared with gpl20/IFN-Υ challenge to the presence of a-bungarotoxin (P>0.05).

The TNF-α released upon this microglial activation plays a central and multifaceted role in affected individuals. Along with IL-1 β, it feeds back to up-regulate microglial release of the EAA, L-cysteine. In turn L-cysteine binds neuronal NMDA receptors thereby lowering the threshold of activation in synergy with HIV-1 gpl20. This then proceeds to a state of excitito-toxicity in which uncontrolled amounts of calcium enter the cell with ensuing activation of the apoptotic cycle. (Garden, Glia 40, 240-251 (2002)).

Another function of the microglial release of TNF-α is to act in synergy with stromal derived factor-1 (SDF-1) to pontentiate glutamate release from neighboring microglia and astrocytes (Id.). Studies have indicated that Platelet-activating factor (PAF) is also released from microglia in response to TNF-α (Id.). In addition TNF-α acts in synergy with the HIV protein Tat, to signal apoptosis in neurons (Id.). Finally, TNF-α can directly activate the neuronal apoptotic pathway by promoting the aggregation of TNF-α receptor-1 (TNFR1) subsequently leading to the activation of caspase-8. TNFR1 is found on a portion of neurons, making the TNF-α induced apoptosis scenario quite likely in the setting of elevations of this cytokine as is seen in HAD (Id.). Neutralization of TNF-α prevents HIV-1 gpl20-induced neurotoxicity in mixed cerebrocortical cultures.

Nitric oxide is also important in the pathophysiology of HAD. It is thought to be related to: impairment of antiviral defense mediated by T-helper-1 immune response by suppressing T-helper-1 functions; inducement of cytotoxic effects by oxidative injury with cellular and organ dysfunctions; and inducement of oxidative stress leading to rapid viral evolution with production of drug-resistant and immunologically tolerant mutants. (Torre et al. Lancet Inf. Dis. 2, 273-280 (2002). Findings also strongly suggest a synergistic attenuation of microglial NO and TNF-α release by pretreatment with galantamine and nicotine. Release of cytokines was significantly less when both medications were added than each was added individually (FIG. 5A and C).

The mechanism of this attenuation relies on α7 nAChR signaling. Data show that co-pretreatment of these cells with α-bungarotoxin (a specific inhibitor of the α7 nAChR) significantly attenuates the effect of galantamine/nicotine on inhibition of microglial TNF-α production and NO release induced by HIV-1 gpl20 and IFN-Υ challenge (Id.) (FIG. 5B and D). The α7 nAChR attenuates cytokine release intra-cellularly through negative modulation of p44/42 MAPK phosphorylation. This is evidenced by and concurrent decreased attenuation of cytokine release in the presence of α-bungarotoxin as well as band density ratio with and without the addition of α-bungarotoxin. As indicated in FIG. 6D, band density ratio of phospho-p44 to total p44 increased from approximately 0.45 to 0.90 in the presence of α-bungarotoxin. This is nearly equal to previous levels when only HIV-1 gpl20 and IFN-Υ were added to microglia. This increased release of cytokines in the presence of HIV-1 gp120, IFN-Υ, and selective α7 nAChR blockade strongly suggests the protective effects mediated by activation of this receptor by galantamine and nicotine. This is in concordance with previous unpublished data indicating that microglial α7 nAChR were responsible for attenuation of cytokine release when they were bound by acetylcholine (Shytle et al.). Further support comes in the form of numerous other studies showing the neuroprotective effects of this receptor when expressed by neurons. (See Kaneko et al., Brian Res., 765, 135-140 (1997).

It will be seen that the objects set forth above, and those made apparent from the foregoing description, were efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims were intended to cover all of the generic and specific features of the invention herein described, and all statements of the scope of the invention which, as a matter of language, might be the to fall therebetween. Now that the invention has been described,

What is claimed is:

1. A method of treating a subject afflicted with a neurodegenerative disorder, comprising the step of concomitantly administering a therapeutically effective amount of a cholinergic agonist and a cholinesterase inhibitor to the subject.

2. The method of claim 1 wherein the neurodegenerative disorder is selected from the group consisting of HIV-associated dementia (HAD) and Alzheimer's disease (AD).

3. The method of claim 1 wherein the administration of the cholinergic agonist and acetylcholinesterase inhibitor is directed to a microglia target cell.

4. The method of claim 1 wherein the cholinergic agonist is nicotine.

5. The method of claim 4 wherein nicotine is administered in concentrations of about 0.3 μM to 5 μm.

6. The method of claim 1 wherein the cholinesterase inhibitor is galantamine.

7. The method of claim 6 wherein galantamine is administered in concentrations of about 0.05 μM to 10 μM.

8. A method of treating a subject afflicted with a neurodegenerative disorder, comprising the step of concomitantly administering to the subject a therapeutically effective amount of nicotine and galantamine, wherein the target cell is a microglia.

9. The method of claim 8 wherein nicotine is administered in concentrations of about 5 μm.

10. The method of claim 8 wherein galantamine is administered in concentrations of about 0.05 μM and 10 μM.

11. A method of modulating microglial activation in a subject, comprising the step of concomitantly administering to the subject a therapeutically effective amount of a cholinergic agonist and a cholinesterase inhibitor.

12. The method of claim 11 wherein the subject suffers from a condition seleceted from the group consisting of HIV-associated dementia (HAD) and Alzheimer's disease (AD).

13. The method of claim 11 wherein the cholinergic agonist is nicotine.

14. The method of claim 13 wherein nicotine is administered in concentrations of about 0.3 μM to 5 μm.

15. The method of claim 11 wherein the cholinesterase inhibitor is galantamine.

16. The method of claim 15 wherein galantamine is administered in concentrations of about 0.05 μM and 10 μM.

17. A method of modulating microglial activation in a subject, comprising the step of concomitantly administering to the subject a therapeutically effective amount of nicotine and galantamine.

18. The method of claim 17 wherein nicotine is administered in concentrations of about 0.3 μM to 5 μm.

19. The method of claim 17 wherein galantamine is administered in concentrations of about 0.05 μM and 10 μM.
